(19) United States
(12) Patent Application Publication
(10) Pub. No.: US 2016/0278710 A1
(43) Pub. Date: Sep. 29, 2016

(54) Noise reduction in an electrophysiological signal

(71) Applicant: Phoenix Research Labs, Pleasanton, CA (US)

(72) Inventors: N.A. (Bert) Massie, San Ramon, CA (US); Jungtae Rha, Pleasanton, CA (US)

(21) Appl. No.: 14/671,483

(22) Filed: Mar. 27, 2015

(51) Int. Cl. A61B 5/00; A61B 5/0496

(52) U.S. Cl. CPC A61B 5/7203; A61B 5/0496; A61B 5/7257; A61B 5/6821

(57) ABSTRACT

Physiological measuring instruments can detect and produce an electrophysiological signal from the physiological response of a biological subject (e.g., a human, an animal, or the like) to stimulus. For example, an electrode in electrical contact with an eye of a human or an animal can detect and produce an electrophysiological signal from the biological response of the eye to light stimulus. One or more noise components in the electrophysiological signal can be reduced by estimating the noise component(s) and then subtracting the estimated noise component(s) from the detected electrophysiological signal.

### BACKGROUND

One challenge in measuring the electrical response of the eye to a flash of light is to obtain a signal that is free from the interfering effects of electrical noise. These electrophysiological measurements are configured differently depending on what signals are to be measured and the type of optical stimulation. For example there is Electroretinogram (ERG), Pattern ERG or PERG and so forth. But they all have the common challenge of accurately measuring signals in the hundred microvolt or lower level against these various sources of interfering noise.

For eye research involving rodents and other animals a typical configuration of electrodes is shown in FIG. 1A. The animal, in this example a rodent, is anesthetized. A test electrode 1 is contacted to the cornea, a reference electrode 2 to the scalp, and a ground electrode 3 to the tail. A flash of light 7 is injected into the eye and onto the retina and the electric signal from the retina is received and recorded as shown in FIG. 1B.

In FIG. 1B is shown the signal receiving system consisting of an instrumentation amplifier 5 which is connected to the test signal 1, the reference signal 2 and the ground signal 3. The analogue to digital converter 4 digitizes the signal and transfers its digital representation 6 to a computer for storage. The foregoing is but an example. There are a variety of connection schemes for the electrodes similar in principle to the one shown in FIG. 1C. Similar to FIG. 1A, a test electrode 8 is attached to the cornea, a reference electrode 9 attached to the cheek, and a ground electrode 10 attached to the scalp (or for example the ear or wrist). The signal reception is identical to FIG. 1B.

In general there are three sources of noise to be considered and they have different characteristics. First, random noise, which is incoherent, is reduced to a generally insignificant level with proper amplifier, electrodes, and signal conditioning. For example, RMS noise in the order of several microvolts is typical whereas the signals of interest can range to hundreds of microvolts peak to peak.

Second, and more problematical, there is pickup of unwanted electrical interference from the main power supply to the facility, referred to as “mains,” and, sometimes other electrical equipment. Mains usually includes the 60 Hz generated by the main electrical power to the facility and 120 Hz from equipment such as fluorescent lamps. However, this signal can be at frequencies other than 60 Hz in some clinics and laboratories, especially internationally, the mains frequency can be as low as 50 Hz. And some power supplies can for example generate more complex interfering signals at other frequencies such as for example 240 Hz.

Third, there can be biological signals picked up such as the heartbeat and breathing. While the heartbeat is not a signal easily characterized as a Fourier series, it does have a stable shape and over periods of time longer than the test time it is usually stable in frequency, amplitude, and phase. Finally, the breathing of the animal or subject can also create interfering signals. However, these signals can also be aperiodic further confounding techniques to remove the breathing interference from the measurements.

These interfering signals can in many instances have levels high enough to materially reduce the integrity of electrophysiology signal. For instance interfering levels in the hundreds of microvolts are possible. And, this may vary with the exact location of the equipment even within the same room. Some researchers mark a place in the laboratory and orientation of the equipment for best results. As a result a robust means for removing this interference would be of great value in research and in the clinic.

Current techniques for mitigation are not robust and frequently inadequate. As a first mitigation reduction of mains pickup is accomplished using a specialized electronic circuit called an instrumentation amplifier. This design attempts to eliminate the mains signal by picking up the interference on both the signal and reference electrodes and then removing the interference by subtraction. While this is beneficial it sometimes does not provide sufficient common mode rejection and does not remove the heartbeat or breathing artifacts.

Another but extreme mitigation against mains pick on the research side is to place the animal and equipment inside a “Faraday cage.” This is a bulky and expensive electrically conductive cage that surrounds the equipment and blocks the interference of the mains. While this is a solution except for biological signals such as the heartbeat or breathing it is quite expensive and is seldom employed.

Yet another technique is to repetitively flash the light and average the signal. The interfering signal will be reduced on the average by the inverse square root of the number of traces averaged. So, for nine traces the pickup is reduced on the average by three times. Sometimes this is not sufficient using a reasonable number of flash repetitions. And this technique is quite objectionable to a patient as the exam and light can be quite uncomfortable and especially objectionable with pediatric and old-aged patients.

The averaging technique may completely fail in the research setting where it is necessary to measure the signal from a totally dark adapted animal; the so-called scotopic regime of retinal response. To maintain the best dark adaptation sometimes animals are placed in the dark for at least twelve hours and the first flash may terminate this total dark adaptation.

Some have elected to use a notch filter to remove the 60 or 120 Hz component of the signal. This is a very poor solution as there is signal information in both of these bands so it is difficult to use this to remove these interferences without degrading the results. And, the precise frequency of the mains varies leaving a standard design with a fixed center frequency for the notch an unacceptable solution.

Finally there is interference from the heartbeat of the animal or patient but this is infrequently a problem in the clinic. Essentially when obtaining a signal representing the eye on animals it can be difficult to place electrodes such that the heart beat is not recorded along with the electrophysiological signal thus reducing the fidelity of the measurement.

All of these issues point to the need for a robust means to eliminate the corrupting signal pickup with special focus on the mains but also on heartbeat and breathing pick-up. By robust it is meant that the signal in all but the absolute worst environments will not be corrupted by either heart beat or breathing or mains pickup and all these without the expense, trouble and bulk of a Faraday cage (which does not eliminate the heart beat or breathing pick-up) and without averaging.

A robust means would remove these artifacts while preserving dark adaptation in research setting would have a substantial impact on eye research. And in the clinical setting many times patients have to be tested more than once to obtain useful results. Techniques to reduce this stress on the patient and the clinical would be very welcome.

### SUMMARY

In some embodiments of the invention, a process for reducing noise in an electrophysiological signal can comprise stimulating a physiological response in a subject, and sensing with an electrode in contact with the subject an electrophysiological signal produced by the subject in response to the stimulating. The process can also include estimating a noise component of the electrophysiological signal, and subtracting the estimated noise component from the electrophysiological signal.

In some embodiments of the invention, a noise reduction electronic apparatus can comprise an electrode, an estimating circuit, and a noise reduction circuit. The electrode can be configured to sense a physiological response of a subject to a stimulus and produce an electrophysiological signal from the sensed response. The estimating circuit can be configured to estimate a noise component of the electrophysiological signal, and the noise reduction circuit can be configured to subtract the estimated noise component from the electrophysiological signal.

### BRIEF DESCRIPTION OF THE DRAWINGS

FIG. 1A: Depiction of typical electrode placement for sensing a physiological response of the eye of a rodent to light.

FIG. 1B: Depiction of typical instrumentation amplifier.

FIG. 1C: Depiction of typical electrode placement for sensing a physiological response of a human eye to light.

FIG. 2: Illustration of an example of a noise reduction circuit.

FIG. 3A: Depiction of a noise signal.

FIG. 3B: Depiction of the noise signal and a sensed physiological signal.

FIG. 3C: Depiction of the combined noise and sensed physiological signal from which an estimated component of the noise signal has been removed.

FIG. 4: Illustration of another example of a noise reduction circuit.

FIG. 5: Depiction of a recorded rodent heartbeat electrical pattern vs. a model function.

FIG. 6: Shows yet another example of a noise reduction circuit.

FIG. 7A: Example depiction of a physiological signal detected from an eye of a mouse in response to a stimulus having a 60 Hz noise signal at about an 80 microvolt level peak to peak.

FIG. 7B: The physiological signal of FIG. 7A from which an estimate of the 60 Hz signal has been subtracted.

FIG. 8: An example of a process that can be performed with and/or by a noise reduction circuit.

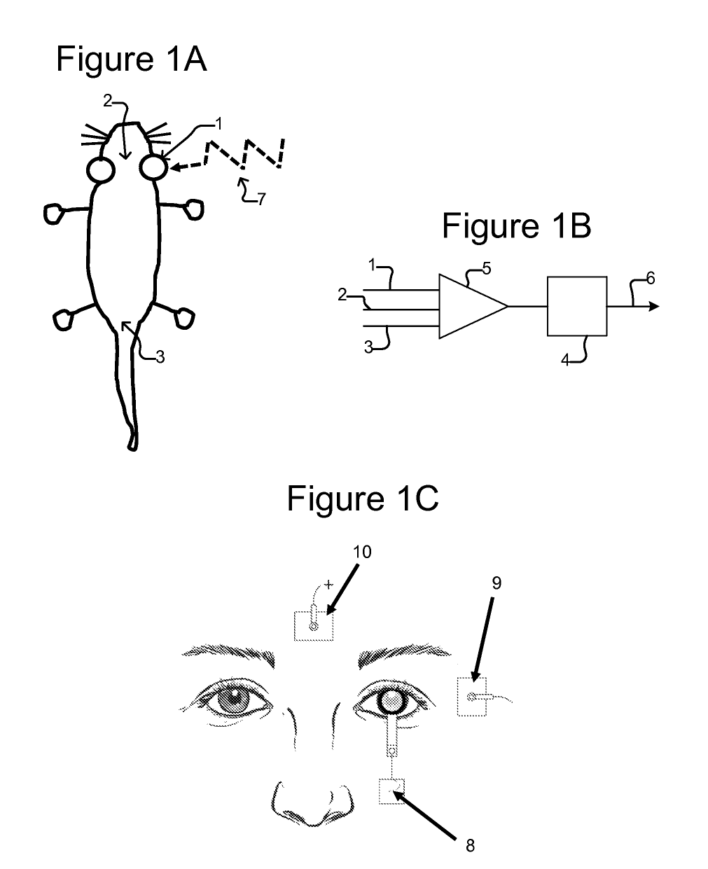

### DETAILED DESCRIPTION OF EXEMPLARY EMBODIMENTS

This specification describes exemplary embodiments and applications of the invention. The invention, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent. The term “ones” means more than one. The term “disposed” encompasses within its meaning “located.”

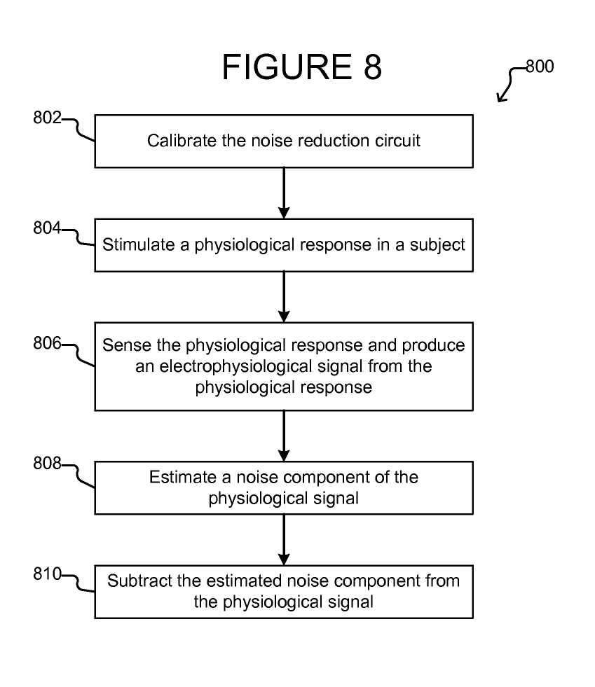

As used herein, “circuit” encompasses within its meaning an electronics module comprising one or a combination of the following configured to perform one or more processes (e.g., the process 800 of FIG. 8), steps of a process (e.g., one or more of the steps of the process 800 of FIG. 8), functions, or the like: (1) a digital memory device for storing non-transitory machine executable instructions (e.g., microcode, firmware, software, or the like) and a digital processing device (e.g., a microprocessor, microcontroller, computer, or the like) for operating in accordance with the machine executable instructions stored in the digital memory device to perform the one or more processes, steps of a process, and/or functions; (2) hardwired digital logic circuitry and/or analog circuitry configured to perform the one or more processes, steps of a process, and/or functions; or (3) a combination of the foregoing configured to perform the one or more processes, steps of a process, and/or functions. A “circuit” can also include analog electronic components.

Physiological measuring instruments can detect and produce an electrophysiological signal from the physiological response of a biological subject (e.g., a human, an animal, or the like) to stimulus. For example, an electrode in electrical contact with an eye of a human or an animal can detect and produce an electrophysiological signal from the biological response of the eye to light stimulus. Some embodiments of the invention can reduce one or more noise components in the electrophysiological signal by estimating the noise component(s) and then subtracting the estimated noise component(s) from the detected electrophysiological signal. For example, some embodiments of the invention can estimate a noise component in the electrophysiological signal from signals detected on the electrode during a time period that the stimulus is not being applied to the subject. As another example, some embodiments of the invention can estimate a noise component of the electrophysiological signal from a noise signal detected at an antenna disposed in proximity to the electrode. As still another example, some embodiments of the invention can estimate a noise component of the electrophysiological signal from noise signals detected at a second electrode electrically connected to the subject at a location not exposed to the stimulus.

Hereinafter, an electrophysiological signal detected on an electrode in contact with a subject in response to a stimulus is denoted S(t), an estimated noise component of the detected signal S(t) is denoted Iₑₛₜ(t), and a noise-reduced version of S(t) resulting from subtraction of Iₑₛₜ(t) from S(t) is denoted E(t).

The electrophysiological signal S(t) on the electrode can be written in terms of its components: S(t)=Ran(t)+E(t)+I(t), where Ran(t) is random low level amplifier and/or thermal noise, I(t) is a noise component or components as discussed above, and E(t) is a noise-reduced version of the measured electrophysiological signal S(t) also as discussed above. Ran(t) is typically small enough to be ignored and is ignored herein. The noise component(s) I(t) can include noise from sources such as ambient power sources or delivery mechanisms (hereinafter “mains” noise), noise from biological functions (e.g., heartbeat, breathing, or the like) of the subject that are not directly related to the electrophysiological signal, or similar sources that produce noise in the electrophysiological signal S(t).

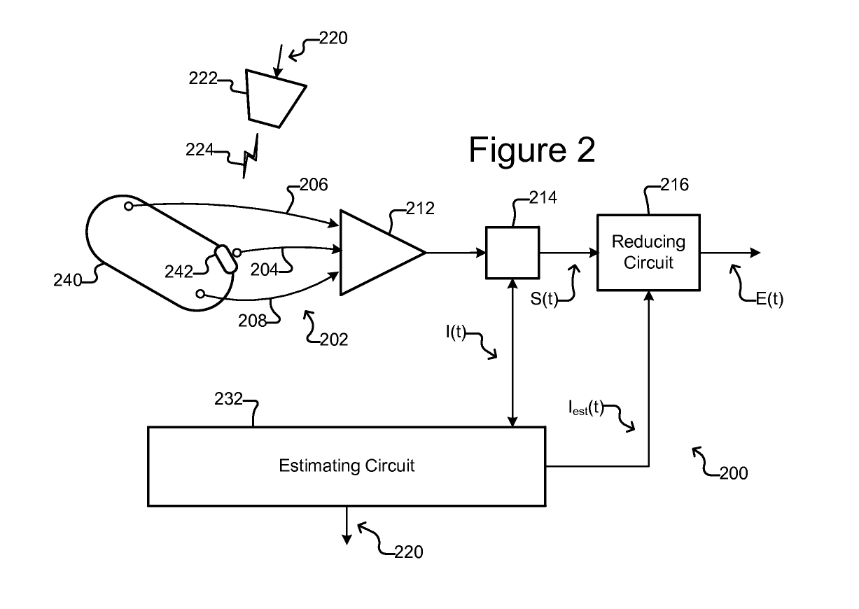

FIG. 2 illustrates a first example of a noise reduction circuit 200, which as shown, can comprise an electrophysiological signal pickup electrode 202, an amplifier 212 (e.g., an instrumentation amplifier), a switch 214, an estimating circuit 232, and a reducing circuit 216. The electrode 202 can comprise a signal electrode element 204 (e.g., a test electrode element) electrically connected to (e.g., in physical contact with) a first location 242 of a subject 240. As shown, the electrode 202 can comprise additional electrode elements such as a ground electrode element 206, a reference electrode element 208, or the like. The subject 240 can be a human, an animal, or the like. The first location 242 can be an organ, such as an eye, of the subject 240. A stimulus source 222 can direct a stimulus 224 onto the first location 242, which can cause an electrophysiological response of the subject 240 at the first location 242. The electrode 202 can convert the electrophysiological response into an electrophysiological signal S(t), which can be amplified by the amplifier 212 and provided through the switch 214 to the reducing circuit 216. The stimulus 224 can be, for example, light. The stimulus source 222 can thus be a light source and can be controllable (e.g., via a control signal 220) to provide a short flash of light, a sequence of repeated short flashes (e.g., a strobe) of light, or a continuous light for a time period to the first location 242 of the subject 240.

The switch 214 can be controllable to switch the output of the amplifier 212 between the reducing circuit 216 and the estimating circuit 232. During a time period in which the stimulus 224 is not provided to the subject 240, the subject 240 does not produce a physiological response to the stimulus 224. Consequently, the signal produced by the electrode 202 and output by the amplifier 212 in the absence of the stimulus 224 is due solely to noise, which can be from any of several possible sources. For example, a noise signal (hereinafter referred to as I(t)) picked up by the electrode 202 in the absence of the stimulus 224 can be mains noise, noise from biological functions of the subject 240 other than the physiological response to the stimulus 224, or the like. Examples of such biological functions include the breathing and/or heartbeat of the subject 240. Regardless of the source of the noise signal I(t) picked up by the electrode 202 in the absence of the stimulus 224, the switch 214 can be set to switch the noise signal I(t) to the estimating circuit 232 rather than the reducing circuit 216. The estimating circuit 232 can then utilize the noise signal I(t) to estimate one or more noise components Iₑₛₜ(t) of the noise signal I(t). Then, while the stimulus 224 is directed to the first location 242 causing the subject to produce the electrophysiological signal S(t), the electrophysiological signal S(t) can be provided to the reducing circuit 216 by the switch 214, and the estimating circuit 232 can provide the estimated noise components Iₑₛₜ(t) to the reducing circuit 216, which can subtract the estimated noise components Iₑₛₜ(t) from the electrophysiological signal S(t), producing the reduced-noise electrophysiological signal E(t).

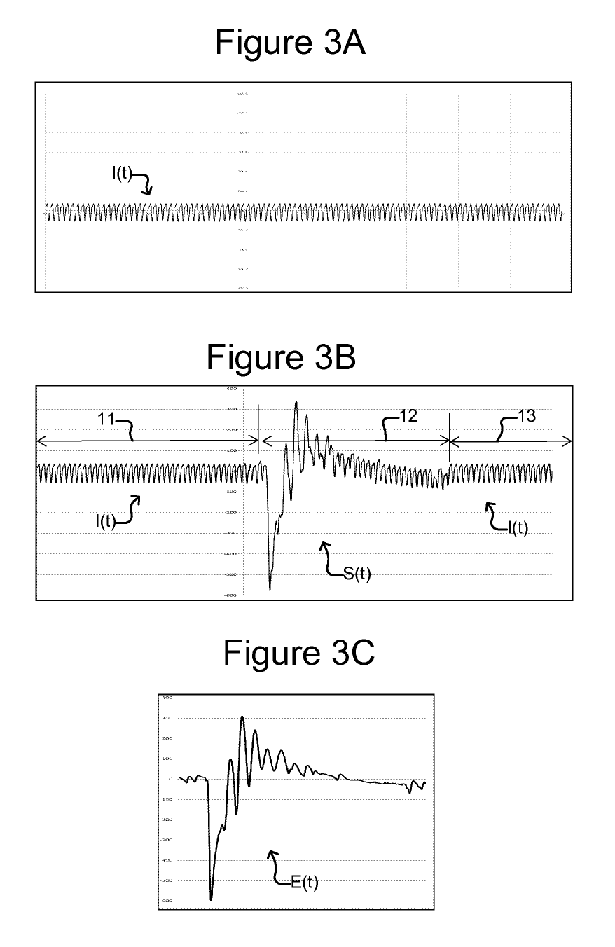

The foregoing operation of the circuit 200 can be performed in any of a number of difference possible sequences of steps. FIG. 3B illustrates an example in which stimulus 224 is provided to the first location 242 of the subject 240 in a time period 12 during which the electrode 202 detects the physiological response of the subject 240 to the stimulus 244 and produces the electrophysiological signal S(t). FIG. 3A illustrates the noise signal I(t); FIG. 3B shows the output of the amplifier 212 during a pre-stimulus time period 11 before the stimulus 224 is directed to the first location 242, a measurement time period 12 in which the stimulus 224 is directed to the first location 242 and the resulting electrophysiological signal S(t) is measured, and a post-stimulus time period 13 when the stimulus 224 is turned off and measurement of the electrophysiological signal S(t) has ceased; and FIG. 3C shows the noise-reduced electrophysiological signal E(t).

During the pre-stimulus period 11, the circuit 200 (e.g., the estimating circuit 232) can turn the stimulus source 222 off and set the switch 214 to connect the output of the amplifier 212 to the estimating circuit 232. The estimating circuit 232 can then receive and utilize the noise signal I(t) to determine the estimated noise components Iₑₛₜ(t) as discussed above. The estimating circuit 232 can then store the estimated noise components Iₑₛₜ(t). At the start of the measurement period 12, the circuit 200 can turn the stimulus source 222 on and set the switch 214 to connect the output of the amplifier 212 to the reducing circuit 216. As the electrode 202, amplifier 212, and switch 214 provide the electrophysiological signal S(t) to the reducing circuit 216, the estimating circuit 232 can provide the stored estimated noise components Iₑₛₜ(t) to the reducing circuit 216, which can subtract the estimated noise components Iₑₛₜ(t) from the electrophysiological signal S(t), producing the reduced-noise electrophysiological signal E(t).

The foregoing operation of the circuit 200 is but an example, and other sequences of steps of the operation are possible. For example, at the start of the pre-stimulus period 12, the circuit 200 can turn the stimulus source 222 on and set the switch 214 to connect the output of the amplifier 212 to the reducing circuit 216. As the electrode 202, amplifier 212, and switch 214 provide the electrophysiological signal S(t) to the reducing circuit 216, the reducing circuit 216 can store the electrophysiological signal S(t). Then, during the post-stimulus period 13, the circuit 200 can turn the stimulus source 222 off and set the switch 214 to connect the output of the amplifier 212 to the estimating circuit 232. The estimating circuit 232 can then receive and utilize the noise signal I(t) to determine the estimated noise components Iₑₛₜ(t) as discussed above. The estimated noise components Iₑₛₜ(t) can then be provided to the reducing circuit 216, which can subtract the estimated noise components Iₑₛₜ(t) from the stored electrophysiological signal S(t), producing the reduced-noise electrophysiological signal E(t).

As yet another example of the sequence of steps of operation of the circuit 200, the circuit 200 can determine first estimated noise components Iₑₛₜ(t) during the pre-stimulus period 11 in FIG. 3B as discussed above, then detect the electrophysiological signal S(t) during the measurement period 12, and then determine second estimated noise components Iₑₛₜ(t) during the post-stimulus period 13. The circuit 200 can thereafter utilize both the first estimated noise components Iₑₛₜ(t) and the second estimated noise components Iₑₛₜ(t) to reduce noise in the electrophysiological signal S(t).

Any part of the noise signal I(t) due to the mains can be substantially the same during the three periods 11, 12, and 13 shown in FIG. 3B. Any part of the noise signal I(t) due to the mains can thus be “coherent,” that is, stable in amplitude, frequency, phase, and/or waveform during the relevant periods. In some embodiments, the estimating circuit 232 can determine the estimated noise components Iₑₛₜ(t) by decomposing the noise signal I(t) into one or more constituent frequencies.

A general expression for the noise signal I(t) (which can be repetitive and coherent) can be I(t)=Σₙ₌₁^{N}Aₙ*fₙ(t+Øₙ), where fₙ(t+Øₙ)=fₙ(Tₙ+Øₙ), Aₙ is the amplitude of each component, fₙ is its functional form, and Øₙ is the phase of the signal. This expression is for a signal (e.g., I(t)) that has a repetition time of Tₙ and is the form for signals such as are experienced in the laboratory or clinic and have the property of being repetitive and coherent over the times of interest. A simplified example of a coherent signal can be (t)=A*sin(wt+Ø).

In one approach, the noise signal I(t) can be measured during the pre-stimulus period 11, the post-stimulus period 13, or both time periods 11 and 13. In a first example, the noise signal I(t) detected by the electrode 202 in the absence of the stimulus 224 can be decomposed by the estimating circuit 232 into one or more components, and the frequency, phase, and/or amplitude of one or more of those components can be obtained by performing a Fourier transform on the noise signal I(t). As another example, the frequency, phase, and/or amplitude of one or more components of the noise signal I(t) can be obtained using a model (e.g., a mathematical model) of the noise signal I(t). A Fourier transform may be a more efficient approach when the noise signal I(t) is primarily from the mains. A model may be a more efficient approach when the noise signal I(t) includes noise from biological functions of the subject 240 such as breathing and/or a heartbeat.

A Fourier transform can be applied to the noise signal I(t) and this will give a spectrum of the noise signal I(t), and its amplitude and phase. However, in nearly all applications signals of interest include only the 60 Hz mains and the first harmonic.

Examining only the 60 Hz fundamental, it would be written as follows:

$$f ( t ) = A * sin ( wt + ∅ )$$

$$\begin{matrix}ℱ \{ f ( t ) \} = ℱ ( w ) \\ = \int f ( t ) {e}^{- i wt} d t \\ = \int f ( t ) cos ( wt ) d t - \\ i \int f ( t ) sin ( wt ) d t ,\end{matrix}$$

$$where$$

$$A ( w ) = \int f ( t ) cos ( wt ) d t , \\ and$$

$$B ( w ) = \int f ( t ) sin ( wt ) d t$$

$$Amplitude \text{:} A = \sqrt{{A ( w )}^{2} + {B ( w )}^{2}}$$

$$Phase \text{:} ∅ = {tan}^{- 1} ( \frac{/ B ( w ) /}{/ A ( w ) /} )$$

$$C ( t ) = A sin ( wt + ∅ )$$

$$C ( t ) = A sin ( wt + ∅ )$$

This technique has the distinct advantage of being simple. For example, no additional receive channel need be implemented. Rather, the technique can be implemented in simple software. A Fourier transform can thus be an efficient approach to decomposing the noise signal I(t), particularly when the noise signal I(t) is primarily from the mains. When the noise signal I(t) includes noise from biological functions of the subject 240 such as breathing and/or a heartbeat, however, a model may be a more efficient approach to decomposing the noise signal I(t).

For example, the following equation is an example of a mathematical model of a subject 240 that is a rodent:

$$C ( t ) = A sin ( wt + ∅ ) {e}^{- {( \frac{t - {t}_{0}}{τ} )}^{2}} .$$

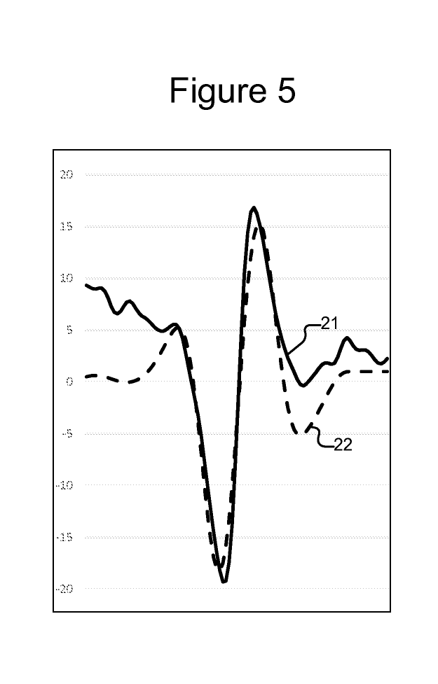

The fit of such a function to an actual recording of a rodent heartbeat is shown in FIG. 5 where trace 21 is heartbeat noise and 22 is the model function. Similar expressions can be written to model a function for noise arising from breathing of the subject 240.

By obtaining the correlation function of C(t) and the trace and varying the time delay between these signals one can find the locations in time of the heart beat and its rate. The functional parameters w, Ø, and τ can then be determined. Finally, A can be determined by subtraction.

In one exemplary embodiment the coherent noise mathematical representation is computed from the measurement of the noise signal I(t) in the pre-stimulus period 11, the post stimulus period 13, or both of the foregoing periods. (See FIG. 3B.) Processing by any of a number of well-known algorithms can determine a function f(t), phase φ, amplitude A and frequency ω of the noise signal I(t). Especially as a result of most common interference comes from the mains and easily represented by one sin function (or perhaps two when the first harmonic is present).

For illustrative purposes a simple case is selected where the coherent portions of the noise signal I(t) can be written as C(t)=A sin (ωt+φ)+B sin (2ωt+β). This would be the expression for 60 Hz and 120 Hz pickup of arbitrary amplitude and phase but related frequencies. Usually however, once ω and φ are known then so is 2 ω and β but B will have to be measured. A noise signal I(t) with more frequency components can be determined as well. Amplitudes (A, B) and phases (φ, β) and frequencies (ω, 2ω) are sufficiently constant over typical times for these experiments and that they readily meet the criterion of coherence. To understand this consideration that the measurement interval for a single electrophysiological signal S(t) is typically 200 milliseconds. For tests with multiple flashes over time the coherence can be checked in between pulses. This is probably not required as the frequency and phase are set by the generating station and are extremely stable. The amplitudes can be checked as the turning on or off of nearby equipment can readily affect this.

When the values of (A,B), (φ, β), and (ω, 2ω) are determined, then the value of S(t) during the measurement time 12 period shown in FIG. 3B can be accurately determined. There are many ways to determine these coefficients and one can include performing a Fourier transform to measure the frequency of the noise signal I(t) and then the phase. The amplitude can be verified by subtraction from the time period 11 (see FIG. 3B) and varying the amplitude A until the net subtracted signal is as low as can be.

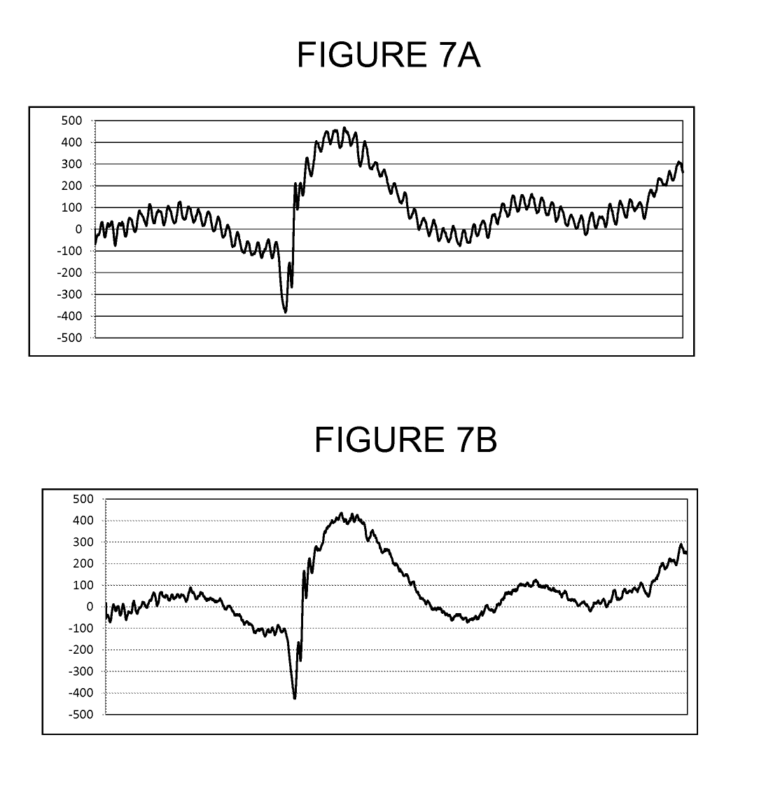

FIG. 7A shows a detected electrophysiological signal S(t) of amplitude peak to valley of around 300 microvolts. This, signal is corrupted by a noise signal I(t) of 60 Hz signal at peak-valley level of 80 microvolts. FIG. 7B shows the result after an estimate of the noise signal Iₑₛₜ(t) is subtracted from the electrophysiological signal S(t).

The estimating circuit 232 and the reducing circuit 216 can be implemented in accordance with the definition of the term “circuit” provided above. That is, the estimating circuit 232 and the reducing circuit 216 can comprise one or a combination of the following configured to perform one or more processes, steps of a process, functions, or the like: (1) a digital memory device for storing non-transitory machine executable instructions (e.g., microcode, firmware, software, or the like) and a digital processing device (e.g., a microprocessor, microcontroller, computer, or the like) for operating in accordance with the machine executable instructions stored in the digital memory device to perform the one or more processes, steps of a process, and/or functions; (2) hardwired digital logic circuitry and/or analog circuitry configured to perform the one or more processes, steps of a process, and/or functions; or (3) a combination of the foregoing configured to perform the one or more processes, steps of a process, and/or functions. The estimating circuit 232 and/or the reducing circuit 216 can thus operate in digital and/or analog domains. Thus, for example, the circuit 200 can be configured so that the noise signal I(t), the electrophysiological signal S(t), and the estimated noise components Iₑₛₜ(t) are in digital or analog format. Thus, although not shown in FIG. 2, the circuit 200 can include analog-to-digital converters (ADCs) and/or digital-to-analog converters (DACs).

The circuit 200 illustrated in FIG. 2 is an example only. Other approaches can utilize different means to measure the noise signal I(t), which can involve different levels of effort and performance.

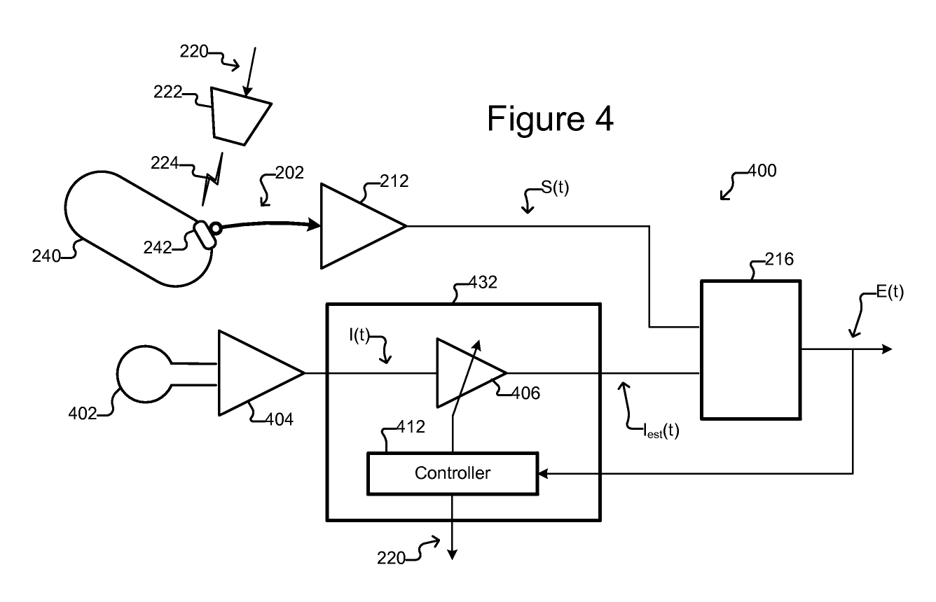

One such approach takes advantage of the fact that there will be a radiated signal from the mains or other external electrical interfering sources even if the key pickup for the subject 240 is by conduction. FIG. 4 illustrates another example of a noise reduction circuit 400, which can include an antenna 402 (e.g., a small coil of wire) for picking up ambient noise. As shown, the circuit 400 can comprise the electrode 202, amplifier 212, and reducing circuit 216 of FIG. 2. The subject 240 and stimulus source 222 can also be the same as shown in FIG. 2 and discussed above. Although not shown in FIG. 4, the electrode 202 can comprise multiple electrode elements as shown in FIG. 2.

As shown in FIG. 4, the antenna 402 can be located in proximity to the subject 240. The antenna 402 can detect ambient noise, and an amplifier 404 (e.g., an instrumentation amplifier) can amplify the detected noise signal I(t). An estimating circuit 432 can determine from the noise signal I(t) estimated noise components Iₑₛₜ(t), which the reducing circuit 216 can subtract from the electrophysiological signal S(t), producing the reduced-noise electrophysiological signal E(t).

In some examples, the antenna 402 can be disposed within ten (10) meters, one (1) meter, five-hundred (500) centimeters, two-hundred (200) centimeters, one-hundred (100) centimeters, fifty (50) centimeters, twenty (20) centimeters, ten (10) centimeters, or five (5) centimeters of the electrode 202. The foregoing numerical ranges are examples only, and the antenna 402 can be more than ten (10) meters or less than five (5) centimeters from the electrode 202.

The antenna 402 can pick up and provide as the noise signal I(t) an accurate copy of noise that is present in the laboratory. The functional shape but not the amplitude, however, of the noise signal I(t) produced by the antenna 402 will be the same as the noise signal from ambient noise picked up by the electrode 202 and thus present in the electrophysiological signal S(t). The amplitude of the noise signal I(t) produced by the antenna 402 can be adjusted as follows, which can thus be a calibration of the circuit 400.

The estimating circuit 432 can comprise a variable gain amplifier (VGA) 406 and a controller 412. The controller 412 can calibrate the gain of the VGA 406 by initially turning off the stimulus source 222. In the absence of the stimulus 224, the signal picked up by the electrode 202 and provided through the amplifier 212 to the reducing circuit 216 will be noise. Assuming that the noise is predominately ambient noise from, for example, the mains, the noise signal I(t) picked up by the antenna 402 should be substantially equal to the noise picked up by the electrode 202. The gain of the VGA 406 can be adjusted until the output of the VGA 406 presented to the reducing circuit 216 is substantially equal to the noise signal from the amplifier 212, which results in the output of the reducing circuit 216 being substantially zero. The circuit 400 can thus be calibrated by: turning the stimulus source 222 off and adjusting the gain of the VGA 406 until the output of the reducing circuit 216 is substantially zero. The foregoing calibration process can be performed automatically by the controller 412, manually by a human user, or a combination of automatically and manually. The controller 412 can be a “circuit” as that term is defined above.

After being calibrated, the circuit 400 can be operated as follows. The circuit 400 (e.g., the controller 412 in the estimating circuit 432) can turn the stimulus source 222 on. The electrode 202 can then pick up and the amplifier 212 amplify an electrophysiological signal S(t) produced by the subject 240 in response to the stimulus 224. Simultaneously, the antenna 402 can pick up ambient noise and produce therefrom a noise signal I(t), which is amplified by the VGA 406 in accordance with its gain as set during the above-described calibration process. The output of the VGA 406, which is labeled estimated noise components Iₑₛₜ(t) in FIG. 4, can then be subtracted from the electrophysiological signal S(t) to produce the reduced-noise electrophysiological signal E(t).

Like the estimating circuit 232 of FIG. 2, the estimating circuit 432 can be implemented in accordance with the definition of the term “circuit” provided above. The estimating circuit 432 and/or the reducing circuit 216 of FIG. 4 can thus operate in digital and/or analog domains. Thus, for example, the circuit 400 can be configured so that the noise signal I(t), the electrophysiological signal S(t), and the estimated noise components Iₑₛₜ(t) are in digital or analog format. Thus, although not shown in FIG. 4, the circuit 400 can include analog-to-digital converters (ADCs) and/or digital-to-analog converters (DACs).

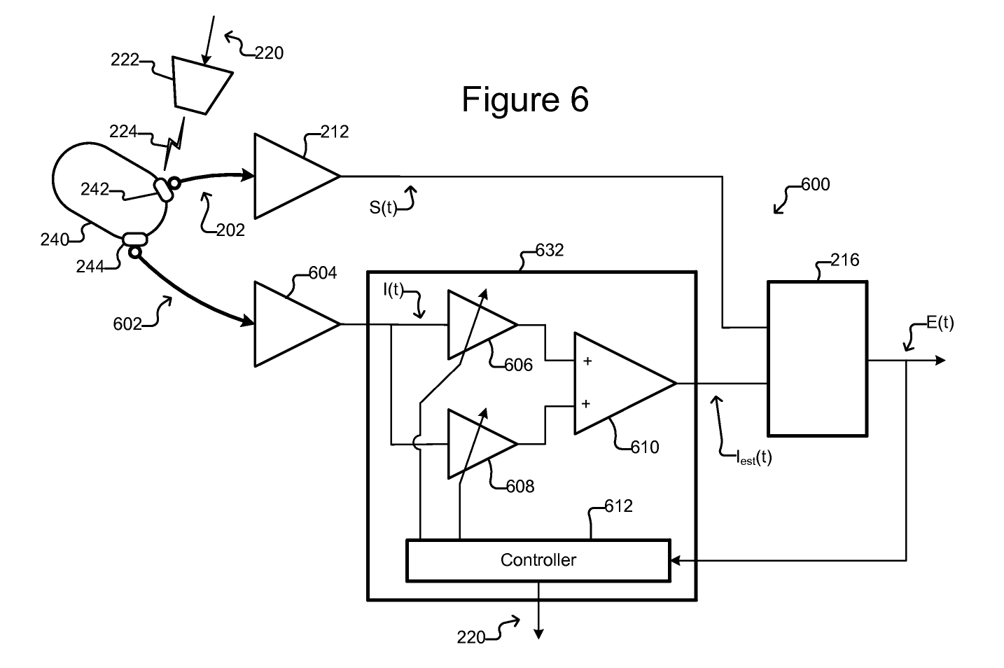

The circuit 400 can efficiently remove noise in the form of ambient noise such as noise from the mains. FIG. 6 illustrates another embodiment of a noise reduction circuit 600 that can also efficiently remove noise from biological functions (e.g., breathing, heartbeat, and the like) of the subject 240.

As shown in FIG. 6, the circuit 600 can comprise the electrode 202, amplifier 212, and reducing circuit 216 of FIG. 2 and discussed above. The circuit 600 can, however, also include a second electrode 602, an amplifier 604 (e.g., an instrumentation amplifier), and an estimating circuit 632. As shown, the second electrode 602 can be electrically connected (e.g., in physical contact with) a second location 244 on the subject 240. The stimulus source 222 can be configured or positioned to provide stimulus 224 to the first location 242 but not the second location 244. As noted above, the first location 242 can be an organ (e.g., a first organ) such as an eye (e.g., a first eye) of the subject 240. The second location 244 can be a second organ such as the other eye of the subject 240. The second organ can be the same type of organ as the first organ or a different type of organ.

The second electrode 602 can be like the first electrode 202. For example, although not shown, the second electrode 602 can comprise multiple electrode elements such as a signal electrode element (not shown but can be like the signal electrode element 204 in FIG. 2) connected to the second location 244, a ground electrode element (not shown but can be like the ground electrode element 206 in FIG. 2), and/or a reference electrode element (not shown but can be like the reference electrode element 208 in FIG. 2). Because the second location 244 of the subject 240 is not exposed to the stimulus 224, the signal picked up on the second electrode 602 should be essentially the same as the noise portion of the electrophysiological signal S(t) picked up by the first electrode 202 in response to the stimulus 224. The signal picked up on the second electrode 602 and amplified by the amplifier 604 is thus labeled a noise signal I(t) in FIG. 6. The estimating circuit 632 can determine estimated noise components Iₑₛₜ(t) from the noise signal I(t). The reducing circuit 216 can subtract the estimated noise components Iₑₛₜ(t) from the electrophysiological signal S(t) to produce the reduced-noise electrophysiological signal E(t).

The second electrode 602 can pick up and provide as the noise signal I(t) an accurate copy of ambient noise (e.g., mains noise) and noise from biological functions (e.g., breathing, heartbeat, or the like) of the subject 240. The portion of the noise signal I(t) from the biological functions will typically be at lower frequencies than the portion of the noise signal I(t) from the ambient noise. As shown in FIG. 6, the estimating circuit 632 can comprise a low frequency VGA 606 for amplifying the portion of the noise signal I(t) from the biological functions of the subject 240 and a high frequency VGA 608 for amplifying the portion of the noise signal I(t) from ambient noise. In some examples, the VGA 606 can be configured to amplify frequencies in a first frequency range, and the VGA 608 can be configured amplify frequencies in a second frequency range. In some embodiments, the first frequency range does not overlap the second frequency range. The amplitudes of the VGAs 606 and 608 can be adjusted as follows, which can thus be a calibration of the circuit 600.

In some examples, the second electrode 602 can be disposed within one (1) meter, five-hundred (500) centimeters, two-hundred (200) centimeters, one-hundred (100) centimeters, fifty (50) centimeters, twenty (20) centimeters, ten (10) centimeters, or five (5) centimeters of the electrode 202. The foregoing numerical ranges are examples only, and the second electrode 602 can be more than one (1) meter or less than five (5) centimeters from the electrode 202.

The controller 612 can calibrate the gains of the VGAs 606 and 608 by initially turning off the stimulus source 222. In the absence of the stimulus 224, the signal picked up by the electrode 202 and provided through the amplifier 212 to the reducing circuit 216 will be noise. The noise signal I(t) picked up by the second electrode 602 should be substantially equal to the noise on the electrode 202. The gains of the VGAs 606 and 608 can be adjusted until the output of a combiner 610 (e.g., a digital adder, a summing amplifier, or the like) presented to the reducing circuit 216 is substantially equal to the noise signal from the amplifier 212, which results in the output of the reducing circuit 216 being substantially zero. The circuit 600 can thus be calibrated by: turning the stimulus source 222 off and adjusting the gains of the VGAs 606 and 608 until the output of the reducing circuit 216 is substantially zero. The foregoing calibration process can be performed automatically by the controller 612, manually by a human user, or a combination of automatically and manually. The controller 612 can be a “circuit” as that term is defined above.

After the circuit 600 is calibrated, it can be operated as follows. The circuit 600 (e.g., the controller 612 in the estimating circuit 632) can turn the stimulus source 222 on. The electrode 202 can then pick up and the amplifier 212 amplify an electrophysiological signal S(t) produced by the subject 240 in response to the stimulus 224. Simultaneously, the second electrode 602 can pick up ambient and biological noise and produce therefrom a noise signal I(t). The low frequency VGA 606 can amplify the portion of the noise signal I(t) attributable to biological functions of the subject 240 and the high frequency VGA 608 can amplify the portion of the noise signal I(t) attributable to ambient noise as set during the above-described calibration process. The outputs of the VGAs 606 and 608 can be summed by the combiner 610. The output of the combiner 610, which is labeled estimated noise components Iₑₛₜ(t) in FIG. 6, can be provided to the reducing circuit 216, which can subtract the estimates of the noise components Iₑₛₜ(t) from the electrophysiological signal S(t) to produce the reduced-noise electrophysiological signal E(t). The circuit 600 can thus efficiently remove both ambient noise and noise from biological functions of the subject 240 from the electrophysiological signal S(t) in response to the stimulus 224.

Like the estimating circuit 232 of FIG. 2 and the estimating circuit 432 of FIG. 4, the estimating circuit 632 can be implemented in accordance with the definition of the term “circuit” provided above. The estimating circuit 632 and/or the reducing circuit 216 in FIG. 6 can thus operate in digital and/or analog domains. Thus, for example, the circuit 600 can be configured so that the noise signal I(t), the electrophysiological signal S(t), and the estimated noise components Iₑₛₜ(t) are in digital or analog format. Thus, although not shown in FIG. 6, the circuit 400 can include analog-to-digital converters (ADCs) and/or digital-to-analog converters (DACs).

FIG. 8 illustrates an example of a process 800 by which any of the circuits 200, 400, or 600 can operate.

As shown, the process 800 can include a step 802 of calibrating a noise reduction circuit. Examples of step 802 include calibrating the circuit 400 or the circuit 600 as discussed above.

At step 804, the process 800 can stimulate a physiological response in a biological subject. This can be accomplished by directing stimulus 224 onto a first location 242 as illustrated in any of FIG. 2, 4, or 6 and discussed above.

At step 806, the process 800 can sense a physiological response of the subject to the stimulus and produce an electrophysiological signal corresponding to the physiological response. This can be performed by the electrode 202 shown in FIGS. 2, 4, and 6 to produce the electrophysiological signal S(t) as discussed above.

At step 808, the process 800 can estimate a noise component of the electrophysiological signal S(t). This can be accomplished by the estimating circuits 232, 432, or 632 producing the estimated noise component(s) Iₑₛₜ(t) as discussed above with respect to FIG. 2, 4, or 6.

At step 810, the process 800 can substrate the estimated noise component(s) Iₑₛₜ(t) from the electrophysiological signal S(t). This can be performed by the reducing circuit 216 subtracting the estimated noise component(s) Iₑₛₜ(t) from the electrophysiological signal S(t) as discussed above with respect to FIG. 2, 4, or 6.

In any of the examples or embodiments discussed above, the electrophysiological signal S(t) can be in any of the following voltage ranges as picked up on the electrode 202 and before being amplified by the amplifier 212: between five (5), ten (10), or twenty (20) microvolts and five-hundred (500), one-thousand (1000), or five-thousand (5000) microvolts. The foregoing numerical ranges are examples only, and the electrophysiological signal S(t) picked up on the electrode 202 before being amplified by the amplifier 212 can be less than five microvolts or greater than five thousand microvolts.

Although specific embodiments and applications of the invention have been described in this specification, these embodiments and applications are exemplary only, and many variations are possible.

We claim:

1. A process of reducing noise in an electrophysiological signal, said process comprising:
stimulating a physiological response in a subject;
sensing with an electrode in contact with said subject an electrophysiological signal produced by said subject in response to said stimulating;
estimating a noise component of said electrophysiological signal; and
subtracting said estimated noise component from said electrophysiological signal.

2. The process of claim 1, wherein said stimulating comprises stimulating an eye of said subject with a flash of light.

3. The process of claim 2, wherein:
said electrode is in electrical contact with said eye, and
said electrophysiological signal is between five and one-thousand microvolts.

4. The process of claim 1, wherein said estimating comprises, over a time period before or after said stimulating, sensing with said electrode noise produced in the absence of said stimulating, said electrode producing a noise signal from said sensed noise.

5. The process of claim 4, wherein:
said estimating further comprises identifying coherent components of said noise signal, and
said subtracting comprises subtracting said coherent components of said noise signal from said electrophysiological signal.

6. The process of claim 4, wherein said estimating further comprises decomposing said noise signal into one or more constituent frequencies.

7. The process of claim 6, wherein said subtracting comprises subtracting said one or more constituent frequencies from said electrophysiological signal.

8. The process of claim 6, wherein said decomposing comprises, for each said constituent frequency, determining an amplitude and a phase offset of said constituent frequency.

9. The process of claim 8, wherein said subtracting comprises, for each said constituent frequency, subtracting at said phase offset of said constituent frequency said amplitude of said constituent frequency from a corresponding frequency of said electrophysiological signal.

10. The process of claim 8, wherein said decomposing comprises performing a Fourier transform on said noise signal.

11. The process of claim 1, wherein said estimating comprises:
while performing said stimulating step, sensing noise with an antenna disposed in proximity to said electrode, and
said antenna producing a noise signal from said sensed noise.

12. The process of claim 11 further comprising, before said stimulating, calibrating a variable gain amplifier (VGA) configured to amplify said noise signal, said calibrating comprising:
before said stimulating, sensing with said electrode noise produced in the absence of said stimulating, said electrode producing a control noise signal therefrom,
simultaneously sensing noise with said antenna, said antenna producing a calibration noise signal therefrom,
amplifying said calibration noise signal produced by said antenna with said VGA, and
while subtracting said amplified calibration noise signal from said control noise signal, determining a gain of said VGA that results in a difference between said amplified control noise signal and said calibration noise signal being substantially zero, and
setting said VGA to said determined gain.

13. The process of claim 12, wherein said subtracting said estimated noise component from said electrophysiological signal comprises:
amplifying with said VGA set at said determined gain said noise signal produced by said antenna from said sensed noise, and
subtracting said amplified noise signal from said electrophysiological signal.

14. The process of claim 1, wherein:
said electrode is a first electrode, and
said estimating comprises:
while performing said stimulating step, sensing noise with a second electrode in contact with said subject, and
said second electrode producing a noise signal from said sensed noise.

15. The process of claim 14, wherein:
said first electrode is in contact with a first organ of said subject,
said second electrode is in contact with a second organ of said subject, and
said first organ and said second organ are the same type of organ.

16. The process of claim 15, wherein:
said first organ is an eye of said subject, and
said second organ is another eye of said subject.

17. The process of claim 14, wherein said noise comprises noise from breathing or a heartbeat of said subject.

18. The process of claim 14 further comprising, before said stimulating, calibrating a plurality of parallel variable gain amplifiers (VGAs) each configured to amplify said noise signal, wherein said calibrating comprises:
sensing with said first electrode noise produced in the absence of said stimulating, said electrode producing a control noise signal therefrom,
simultaneously sensing noise with said second electrode, said second electrode producing a calibration noise signal therefrom,
amplifying said calibration noise signal with said VGAs, and
while subtracting a sum of outputs of said VGAs from said control noise signal produced by said first electrode, determining a gain for each of said VGAs that results in a difference between said control noise signal and said sum being substantially zero, and
setting each said VGA to its respective determined gain.

19. The process of claim 18, wherein said subtracting said estimated noise component from said electrophysiological signal comprises:
with each said VGA set at its respective determined gain, amplifying said noise signal produced by said second electrode from said sensed noise,
summing outputs of said VGAs, and
subtracting said sum of said outputs of said VGAs from said electrophysiological signal.

20. The process of claim 19, wherein:
one of said VGAs is configured to amplify components of said noise signal produced by said second electrode within a first frequency range,
another of said VGAs is configured to amplify components of said noise signal produced by said second electrode within a second frequency range, and
said first frequency range does not overlap said second frequency range.

21. A noise reduction electronic apparatus comprising:
an electrode configured to sense a physiological response of a subject to a stimulus and produce an electrophysiological signal from said sensed response;
an estimating circuit configured to estimate a noise component of said electrophysiological signal; and
a noise reduction circuit configured to subtract said estimated noise component from said electrophysiological signal.

22. The apparatus of claim 21, wherein said electrode is configured to electrically contact an eye of said subject and produce said electrophysiological signal at a voltage level between five and one-thousand microvolts.

23. The apparatus of claim 21, wherein:
said estimating circuit is configured to estimate said noise component by:
in the absence of said stimulus, capture an output of said electrode as a noise signal, and
in the presence of said stimulus, capture an output of said electrode as said electrophysiological signal; and
said noise reduction circuit is configured to subtract said noise signal from said electrophysiological signal.

24. The apparatus of claim 23, wherein said estimating circuit comprises a switch for activating and deactivating a source of said stimulus to said subject.

25. The apparatus of claim 21, wherein:
said estimating circuit is configured to estimate said noise component by:
in the presence of said stimulus, capture an output of said antenna as a noise signal, and
in the presence of said stimulus, capture an output of said electrode as said electrophysiological signal; and
said noise reduction circuit is configured to subtract said noise signal from said electrophysiological signal.

26. The apparatus of claim 25, wherein said estimating circuit comprises a variable gain amplifier (VGA) configured to amplify said noise signal from said antenna.

27. The apparatus of claim 26 further comprising a calibration circuit configured to:
in the absence of said stimulus, capture an output of said electrode as a control noise signal,
in the absence of said stimulus, capture an output of said antenna as a calibration noise signal,
while said noise reduction circuit subtracts said calibration noise signal from said control noise signal, adjust a gain of said VGA until a difference between said calibration noise signal and said control noise signal is substantially zero, and
set said gain of said VGA to said adjusted gain.

28. The apparatus of claim 21, wherein said electrode is a first electrode configured to contact said subject at a first location, said apparatus further comprising a second electrode configured to contact said subject at a second location.

29. The apparatus of claim 28, wherein:
said estimating circuit is configured to estimate said noise component by, in the presence of said stimulus directed to said first location but not said second location:
capture an output of said first electrode as said electrophysiological signal, and
capture an output of said second electrode as a noise signal; and
said noise reduction circuit is configured to subtract said noise signal from said electrophysiological signal.

30. The apparatus of claim 29, wherein:
said estimating circuit comprises:
a plurality of variable gain amplifier (VGAs) configured to amplify said noise signal from said antenna, and
a combiner configured to combine outputs of said VGAs; and
said noise reduction circuit is configured to subtract an output of combiner from said electrophysiological signal.

31. The apparatus of claim 30 further comprising a calibration circuit configured to:
in the absence of said stimulus, capture an output of said first electrode as a control noise signal,
in the absence of said stimulus, capture an output of said combiner as a calibration signal,
while said noise reduction circuit subtracts said calibration signal from said control noise signal, adjust gains of said VGAs until a difference between said calibration signal and said noise signal is substantially zero, and
set said gain of each said VGA to its respective adjusted gain.
